(19) United States
(12) Patent Application Publication
(10) Pub. No.: US 2024/0182384 A1
(43) Pub. Date: Jun. 6, 2024

(54) Method and Device for Converting Waste Plastic Pyrolysis Oil into Light Olefins with High Yield

(71) Applicants: SK Innovation Co., Ltd., Seoul (KR); SK Geo Centric Co., Ltd., Seoul (KR)

(72) Inventors: Hee Jung Jeon, Daejeon (KR); Ok Youn Kim, Daejeon (KR); Ho Won Lee, Daejeon (KR)

(21) Appl. No.: 18/476,379

(22) Filed: Sep. 28, 2023

(30) Foreign Application Priority Data
Dec. 1, 2022 (KR) 10-2022-0165399

(51) Int. Cl. C07C 4/06; B01D 21/26; B01J 8/24; B01J 8/38; B01J 21/04; B01J 21/16; B01J 29/40; B01J 35/02; B01J 35/08; B01J 37/30; B01J 38/02; B01J 38/12

(52) U.S. Cl. CPC C07C 4/06; B01D 21/267; B01J 8/24; B01J 8/382; B01J 21/04; B01J 21/16; B01J 29/40; B01J 35/023; B01J 35/08; B01J 37/30; B01J 38/02; B01J 38/12; B01J 2208/00761; B01J 2208/00769; B01J 2229/186; C07C 2521/10; C07C 2521/16; C07C 2529/40

(57) ABSTRACT

Provided is a method for converting waste plastic pyrolysis oil into light olefins with a high yield. The method includes (1) putting waste plastic pyrolysis oil into a reactor; (2) allowing the waste plastic pyrolysis oil to react in the reactor in the presence of a catalytic cracking catalyst containing (a) a compound of an alkali metal or a compound of an alkaline earth metal and (b) a zeolite to form a product; and (3) recovering light olefins by separating the catalytic cracking catalyst and oil from the product obtained in step (2).

### CROSS-REFERENCE TO RELATED APPLICATION

This application claims priority to Korean Patent Application No. 10-2022-0165399, filed Dec. 1, 2022, the disclosure of which is hereby incorporated by reference in its entirety.

### BACKGROUND OF THE INVENTION

### Field of the Invention

The following disclosure relates to a method and device for converting waste plastic pyrolysis oil into light olefins with a high yield.

### Description of Related Art

Waste plastics, which are produced using petroleum as a feedstock, are difficult to recycle and are mostly disposed of as garbage. These wastes take a long time to degrade in nature, which causes contamination of the soil and serious environmental pollution. For example, as plastic decomposes by exposure to sunlight and heat, the plastic waste releases greenhouse gases such as methane and ethylene. Incineration of plastic waste releases significant amounts of greenhouse gases (GHG), such as carbon dioxide, nitrous oxide and/or methane, into the environment. Carbon dioxide is the primary greenhouse gas contributing to climate change. Therefore, it is desirable to provide a process for recycling waste plastic to reduce waste and/or ameliorate the release of greenhouse gases into the environment by decomposition and/or incineration of the waste plastic. By recycling, environmental pollution is reduced.

As a method for recycling waste plastics, there is a method for pyrolyzing and converting waste plastics into usable oil, and the obtained oil is called waste plastic pyrolysis oil.

Since waste plastic pyrolysis oil is a mixture of hydrocarbon oils having various boiling points and various molecular weight distributions, and a composition or reaction activity of impurities in the pyrolysis oil varies depending on the boiling point and molecular weight distribution characteristics of the mixture of hydrocarbon oils, the waste plastic pyrolysis oil cannot be directly used in the petrochemical industry or in the field, and needs to go through a separation process by boiling point or a refinery process.

In the mixture of hydrocarbon oils, olefins, for example, light olefins such as ethylene and propylene, have been widely used in the petrochemical industry. Until now, most ethylene or propylene has been mainly produced by pyrolysis of hydrocarbon oils containing a paraffinic compound as a main component, such as natural gas, naphtha oil, and gas oil in a steam atmosphere at a high temperature of 800° C. or higher under a catalyst-free condition, but the method described above has problems of a low conversion rate of hydrocarbons and low olefin selectivity. As a measure to improve reaction efficiency such as a conversion rate of hydrocarbons or olefin selectivity, a fluid catalytic cracking (FCC) process has been performed. Representative examples of the FCC process include a catalytic cracking process using an acid catalyst. Among various acid catalysts, zeolites have been most widely used, and as representative zeolites for catalytic cracking, ZSM-5, USY, REY, β-zeolite, and the like have been used.

However, waste plastic pyrolysis oil contains an excessive amount of impurities such as chlorine, nitrogen, or a metal compared to crude oil, natural gas, and naphtha oil, and in the fluid catalytic cracking process of waste plastic pyrolysis oil, a catalytic active site is neutralized or deactivated by the impurities, and thus the reaction activity is significantly reduced. In the case of waste plastic pyrolysis oil according to the related art, a conversion process has been performed as a method of removing a deactivated waste catalyst in the middle of the process and making-up a new catalyst again, but since the deactivated catalyst contains an excessive amount of impurities and its deactivated amount is significant, serious environmental problems are caused, and there are economic and environmental constraints on its disposal and treatment. In addition, in the process of removing a waste catalyst and making-up a new catalyst, as a heat loss or energy loss occurs, process efficiency is significantly reduced and it is difficult to maintain uniform reaction activity, and as a result, constant quality may not be obtained. The most serious problem is that the conversion yield from waste plastic pyrolysis oil into light olefins is remarkably low, and impurities such as chlorine, nitrogen, or a metal are still contained in the converted light olefins, which makes it difficult to economically and commercially put the converted light olefins into practical use due to deterioration of quality.

Therefore, there is a demand for a technology capable of producing light olefins from waste plastic pyrolysis oil with a high yield in a fluid catalytic cracking process.

### SUMMARY OF THE INVENTION

An embodiment of the present disclosure is directed to providing a method and device for converting waste plastic pyrolysis oil into light olefins with a high yield.

Another embodiment of the present disclosure is directed to providing a method and device for converting waste plastic pyrolysis oil into light olefins with a high yield that may implement a conversion yield from waste plastic pyrolysis oil into light olefins of 10% or more, or 25% or more, or 40% or more.

Still another embodiment of the present disclosure is directed to providing a method and device for converting waste plastic pyrolysis oil into light olefins with a high yield that may stably perform a light olefin conversion process for a long time by preventing catalytic deactivation due to impurities contained in the waste plastic pyrolysis oil.

Still another object of the present disclosure is directed to providing a method and device for converting waste plastic pyrolysis oil into light olefins with a high yield that may minimize a content of impurities in light olefins converted from waste plastic pyrolysis oil.

In one general aspect, a method for converting waste plastic pyrolysis oil into light olefins with a high yield comprises: (1) putting waste plastic pyrolysis oil into a reactor; (2) allowing the waste plastic pyrolysis oil to react in the reactor in the presence of a catalytic cracking catalyst comprising (a) a compound of an alkali metal or a compound of an alkaline earth metal and (b) a zeolite to form a product; and (3) recovering light olefins by separating the catalytic cracking catalyst and oil from the product obtained in the step (2).

In an exemplary embodiment, the compound of the alkali metal or the compound of the alkaline earth metal may comprise an oxide, a hydroxide, a carbonate, a silicate, a sulfate, an acetate, a nitride, or an alkoxide.

In an exemplary embodiment, the compound of the alkaline earth metal may comprise magnesium oxide.

In an exemplary embodiment, the catalytic cracking catalyst may satisfy an Mg/Al molar ratio of 10 to 40.

In an exemplary embodiment, the zeolite may comprise ZSM-5, ZSM-11, Y-zeolite, ferrierite, mordenite, MCM-22, SUZ-4, and/or L-type zeolite.

In an exemplary embodiment, the catalytic cracking catalyst may further comprise clay and a binder.

In an exemplary embodiment, the binder may comprise a silica-based compound and/or an aluminum-based compound.

In an exemplary embodiment, the catalytic cracking catalyst may comprise 1 to 10 wt % of the compound of the alkali metal or the compound of the alkaline earth metal, 20 to 70 wt % of the zeolite, 10 to 60 wt % of the clay, and 10 to 50 wt % of the binder.

In an exemplary embodiment, the catalytic cracking catalyst may have an average particle size of 50 to 2,000 μm.

In an exemplary embodiment, the reactor may comprise a fluidized bed reactor.

In an exemplary embodiment, the waste plastic pyrolysis oil may comprise a vacuum gas oil (VGO) component having a boiling point of 340° C. or higher at atmospheric pressure.

In an exemplary embodiment, the step (2) may be performed at a temperature of 400 to 600° C. and a reaction pressure of 50 to 200 kPa.

In an exemplary embodiment, the method for converting waste plastic pyrolysis oil into light olefins with a high yield may further comprise: (3-1) recovering the catalytic cracking catalyst separated in the step (3), putting the recovered catalytic cracking catalyst into a regenerator, and then oxidizing the compound of the alkali metal or oxidizing the compound of the alkaline earth metal to form an oxidized compound of the alkali metal or an oxidized compound of the alkaline earth metal; and (3-2) circulating the catalytic cracking catalyst comprising the oxidized compound of the alkali metal or the oxidized compound of the alkaline earth metal to the step (2) and re-feeding the catalytic cracking catalyst comprising the oxidized compound of the alkali metal or the oxidized compound of the alkaline earth metal into the reactor.

In an exemplary embodiment, the step (3-1) may be performed at 600 to 800° C. in the presence of oxygen gas.

In an exemplary embodiment, the method for converting waste plastic pyrolysis oil into light olefins with a high yield may further comprise, before the step (3-1), stripping the catalytic cracking catalyst separated in the step (3).

In an exemplary embodiment, in the recovered light olefins, a content of chlorine may be 10 ppm or less and a content of nitrogen may be 50 ppm or less.

In another general aspect, a device for converting waste plastic pyrolysis oil into light olefins with a high yield comprises: a fluidized bed reactor into which waste plastic pyrolysis oil is introduced and in which a catalytic cracking reaction is performed in the presence of a catalytic cracking catalyst comprising (a) a compound of an alkali metal or a compound of an alkaline earth metal and (b) a zeolite to form a product; a cyclone into which the product is introduced from the fluidized bed reactor and in which the product is separated into the catalytic cracking catalyst and oil; and a stabilizer into which the oil is introduced from the cyclone and in which the oil is separated into a gas component and a liquid component.

In an exemplary embodiment, the compound of the alkali metal or the compound of the alkaline earth metal may comprise an oxide, a hydroxide, a carbonate, a silicate, a sulfate, an acetate, a nitride, or an alkoxide.

In an exemplary embodiment, the compound of the alkaline earth metal may comprise magnesium oxide.

In an exemplary embodiment, the device for converting waste plastic pyrolysis oil into light olefins with a high yield may further comprise a regenerator into which the catalytic cracking catalyst is introduced from the cyclone and in which the compound of the alkali metal or the compound of the alkaline earth metal is oxidized, wherein the catalytic cracking catalyst comprising the oxidized compound of the alkali metal or the oxidized compound of the alkaline earth metal is re-introduced into the fluidized bed reactor from the regenerator through a recirculation line.

Other features and aspects will be apparent from the following detailed description, the drawings, and the claims.

### DESCRIPTION OF THE INVENTION

Unless the context clearly indicates otherwise, the singular forms of the terms used in the present specification may be interpreted as including the plural forms. As used herein, the singular form of “a”, “an”, and “the” include plural referents unless the context clearly states otherwise.

For the purposes of this specification, unless otherwise indicated, all numbers expressing quantities of ingredients, reaction conditions, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

A numerical range used in the present specification includes upper and lower limits and all values within these limits, all double limited values, and all possible combinations of the upper and lower limits in the numerical range defined in different forms. Unless otherwise specifically defined in the present specification, values out of the numerical range that may occur due to experimental errors or rounded values also fall within the defined numerical ranges. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

The expression “comprise(s)” described in the present specification is intended to be an open-ended transitional phrase having an equivalent meaning to “include(s)”, “contain(s)”, “have (has)”, or “are (is) characterized by”, and does not exclude elements, materials, or steps, all of which are not further recited herein.

Unless otherwise defined, a unit of “%” used in the present specification refers to “wt %”.

Unless otherwise defined, a “boiling point” used in the present specification is based on atmospheric pressure, and the expression “bp” or the like refers to a boiling point.

Unless otherwise defined, a unit of “ppm” used in the present specification refers to “mass ppm”.

Waste plastic pyrolysis oil is a mixture of hydrocarbon oils produced by pyrolysis of waste plastics, and the waste plastics may be solid or liquid wastes related to synthetic polymer compounds such as waste synthetic resins, waste synthetic fibers, waste synthetic rubber, and waste vinyl. The mixture of hydrocarbon oils may contain impurities such as a chlorine compound, a nitrogen compound, or a compound of an alkali metal or an alkaline earth metal, in addition to the hydrocarbon oils, may contain impurities in the form of compounds in which chlorine, nitrogen, or a metal is bonded to hydrocarbons, and may contain hydrocarbons in the form of olefins. For example, the waste plastic pyrolysis oil may contain 300 ppm or more of nitrogen, 30 ppm or more of chlorine, 30 ppm or more of a metal, 20 vol % or more of an olefin, and 1 vol % or more of a conjugated diolefin. As described above, when a fluid catalytic cracking process is performed on waste plastic pyrolysis oil comprising various impurities by a method according to the related art, reaction activity and reaction yield are significantly reduced due to the impurities, and there is a fatal problem that impurities such as chorine, nitrogen, or a metal are still present in the converted light olefins.

Accordingly, according to a method for converting waste plastic pyrolysis oil into light olefins with a high yield according to the present disclosure, a conversion yield of light olefins from waste plastic pyrolysis oil may be significantly improved, a light olefin conversion process may be stably performed for a long time by suppressing catalytic deactivation due to impurities, and a content of impurities such as chlorine or nitrogen in the converted light olefins may be minimized. That is, the problems of both low yield and impurity content occurring when a feedstock is waste plastic pyrolysis oil may be solved, and high-quality light olefins with minimized impurities may be obtained from the waste plastic pyrolysis oil with a high yield.

The present disclosure provides a method for converting waste plastic pyrolysis oil into light olefins with a high yield, the method comprising: (1) putting waste plastic pyrolysis oil into a reactor; (2) allowing the waste plastic pyrolysis oil to react in the reactor in the presence of a catalytic cracking catalyst comprising (a) a compound of an alkali metal or a compound of an alkaline earth metal and (b) a zeolite to form a product; and (3) recovering light olefins by separating the catalytic cracking catalyst and oil from the product obtained in the step (2).

The waste plastic pyrolysis oil comprises a mixture of hydrocarbon oils, and comprises, for example, a mixture of hydrocarbon oils having various boiling points and molecular weight distributions such as C7 to C9 Naphtha having a boiling point of 80 to 150° C. at atmospheric pressure, C10 to C17 Kerosene having a boiling point of 150 to 265° C., C18 to C20 LGO having a boiling point of 265 to 340° C., and C21 or more VGO/AR having a boiling point of 340° C. or higher. As described below, VGO has conversion efficiency that is much lower than those of Kerosene and Naphtha in a light olefin conversion process according to the related art, making it difficult to use VGO, and therefore, the conversion process has been performed mainly using Kerosene and Naphtha as a feedstock excluding VGO. On the other hand, the light olefin high-yield conversion process of the present disclosure may implement a significantly excellent cracking efficiency and light olefin conversion efficiency even when waste plastic pyrolysis oil comprising a vacuum gas oil (VGO) component is used as a feedstock.

The waste plastic pyrolysis oil comprising a mixture of various hydrocarbon oils may be converted into light olefins with a high yield using the catalytic cracking catalyst comprising (a) the compound of the alkali metal or the compound of the alkaline earth metal and (b) a zeolite. In addition, the compound of the alkali metal or the compound of the alkaline earth metal may prevent deactivation of the catalytic cracking catalyst by trapping impurities contained in the waste plastic pyrolysis oil, such as chlorine, nitrogen, sulfur, and/or oxygen, and may minimize impurities in the converted light olefins.

In an exemplary embodiment, the alkali metal may comprise a metal selected from lithium, sodium, potassium, and/or rubidium. In an exemplary embodiment, the alkaline earth metal may comprise a metal selected from magnesium, calcium, strontium, and/or barium. In some embodiments, considering the ability to trap impurities, the alkali metal may be sodium or potassium, and the alkaline earth metal may be magnesium or calcium.

In an exemplary embodiment, the compound of the alkali metal or the compound of the alkaline earth metal may comprise an oxide, a hydroxide, a carbonate, a silicate, an acetate, a nitride, or an alkoxide of the alkali metal or the alkaline earth metal. As another exemplary embodiment, the compound of the alkali metal or the compound of the alkaline earth metal may have a structure. For example, the compound of the alkali metal or the compound of the alkaline earth metal may have a hydrotalcite-like structure, a double-layer hydroxide structure, a spinel structure, or a perovskite structure. These are merely non-limiting examples, and the present disclosure is not limited thereto. The compound of the alkali metal or the compound of the alkaline earth metal described above may prevent deactivation of the catalytic cracking catalyst by trapping impurities contained in the waste plastic pyrolysis oil, such as chlorine, nitrogen, sulfur, and/or oxygen, and may minimize impurities in the converted light olefins.

Preferably, considering the light olefin conversion efficiency and impurity removal efficiency of the waste plastic pyrolysis oil, the compound of the alkali metal or the compound of the alkaline earth metal may be potassium oxide, calcium oxide, or magnesium oxide, or magnesium oxide. The magnesium oxide has the best ability to trap chlorine among the impurities, such that the magnesium oxide may effectively remove chlorine impurities contained in the waste plastic pyrolysis oil.

In an exemplary embodiment, the zeolite may comprise ZSM-5, ZSM-11, Y-zeolite, ferrierite, mordenite, MCM-22, SUZ-4, and/or L-type zeolite. A zeolite is a porous molecular sieve having a rich pore structure and a large specific surface area, and has a lot of active sites and excellent catalytic cracking efficiency. The zeolite may control the cracking activity according to the number of atoms constituting the pores, a pore size, or a stereostructure (one-dimensional, two-dimensional, or three-dimensional structure) of the pores. Zeolites known in the related art other than the zeolites described above may be used without limitation, but considering characteristics of conversion into light olefins of waste plastic pyrolysis oil, it is preferable to use ZSM-5 or Y-zeolite.

In an exemplary embodiment, the catalytic cracking catalyst may further comprise clay and a binder. As the catalytic cracking catalyst further comprises clay and a binder, the catalytic cracking catalyst may have high mechanical strength and may be used in a large-scale petrochemical process such as a process of upgrading waste plastic pyrolysis oil.

The binder may comprise a silica-based compound and/or an aluminum-based compound. For example, the binder may comprise silica sol, a silicic acid solution, water glass, alumina, alumina-silica, and/or pseudo-boehmite alumina (PBA), but this is only an example and the binder is not limited thereto.

The clay may comprise kaolin or a mixture of kaolin and montmorillonite. In this case, Clay has a characteristic of having a weak acid site and can crack the heavy hydrocarbon at high temperatures. The weak acid site means an acid site at which ammonia can be desorbed at 400° C. or less at ammonia TPD analysis. Also, The clay used is good when the surface area is large and the mesopores are well developed, but the clay of low surface area does not mean that it cannot be used.

In an exemplary embodiment, the catalytic cracking catalyst may comprise 1 to 10 wt % of the compound of an alkali metal or an alkaline earth metal, 20 to 70 wt % of the zeolite, 10 to 60 wt % of the clay, and 10 to 50 wt % of the binder. When the catalytic cracking catalyst satisfies all the above weight ranges, the light olefin conversion yield may be significantly improved, and the catalytic deactivation may be effectively prevented by adsorption of the impurities, and as a result, the fluid catalytic cracking process may be stably performed for a long time.

Each component contained in the catalytic cracking catalyst will be individually described. When the compound of the alkali metal or the compound of the alkaline earth metal is comprised in the above weight range, the light olefin conversion yield and the impurity removal effect may be improved, and the activity of the catalytic cracking catalyst and hydrothermal stability may also be improved. Specifically, the compound of the alkali metal or the compound of the alkaline earth metal may be comprised in an amount of 1 to 8 wt %, or 3 to 6 wt %.

When the zeolite is comprised in the weight range, the catalytic cracking efficiency may be improved, and the light olefin conversion yield may be maximized. In some embodiments, the zeolite may be comprised in an amount of 20 to 60 wt %, or 30 to 50 wt %.

When the clay is comprised in the above weight range, the specific gravity of the catalytic cracking catalyst and the overall activity of the catalytic cracking catalyst may be optimized. In some embodiments, the clay may be comprised in an amount of 10 to 50 wt %, or 20 to 40 wt %.

When the binder is contained in the above weight range, physical properties such as abrasion strength of the catalytic cracking catalyst may be excellently maintained. In some embodiments, the binder may be comprised in an amount of 10 to 40 wt %, or 15 to 35 wt %.

In an exemplary embodiment, the catalytic cracking catalyst may satisfy an Mg/Al molar ratio of 10 to 40. As described above, considering the light olefin conversion efficiency and the impurity removal efficiency, the compound of the alkali metal or the compound of the alkaline earth metal may be magnesium oxide. The magnesium oxide adsorbs impurities such as chlorine, nitrogen, and/or sulfur contained in the waste plastic pyrolysis oil, such that it is possible to prevent a solid acid active site in the zeolite from being deactivated. However, when a content of the magnesium oxide is too high, the activation of zeolite may be interfered with, such that the cracking activity and the light olefin conversion yield may be rather reduced, and when the content of the magnesium oxide is too low, the impurity removal effect may be insufficient. Therefore, when the Mg/Al molar ratio satisfies 10 to 40 by optimizing the molar ratio of Mg in the magnesium oxide to Al in the zeolite, the impurity removal effect and the light olefin conversion yield may be maximized. In some embodiments, the Mg/Al molar ratio may be 10 to 35, or 15 to 30.

In an exemplary embodiment, the catalytic cracking catalyst may have an average particle size of 50 to 2,000 μm. In a case where a catalytic cracking catalyst having the above size is screened and used to correspond to intrinsic properties of waste plastic pyrolysis oil, such as a viscosity and a density, the olefin selectivity and the catalytic activity may be significantly improved. In some embodiments, the average particle size may be 50 to 1,000 μm, or 50 to 700 μm, or 50 to 200 μm, or 80 to 150 μm.

In an exemplary embodiment, the catalytic cracking catalyst may have a total specific surface area of 50 to 150 m²/g and an apparent density of 0.5 to 1 g/cm³. Within the above ranges, a contact area with the feedstock and the cracking efficiency are optimized, such that the light olefin conversion yield may be improved. In some embodiments, the total specific surface area may be 50 to 130 m²/g and the apparent density may be 0.5 to 0.8 g/cm³, or the total specific surface area may be 70 to 100 m²/g and the apparent density may be 0.6 to 0.7 g/cm³.

In an exemplary embodiment, the catalytic cracking catalyst may satisfy the following Expression 1.

2<(D90−D10)/D50<5 [Expression 1]

Expression 1 represents a particle size distribution width of the catalytic cracking catalyst in a volume-based distribution measured with a laser diffraction particle size distribution measuring device, in which D10 is a 10% cumulative diameter, D50 is a 50% cumulative diameter, and D90 is a 90% cumulative diameter.

As the particle size distribution width of the catalytic cracking catalyst satisfies Expression 1, a structural collapse of the zeolite due to steam may be prevented, such that catalytic stability may be improved. As described below, the catalytic cracking catalyst is used in both a reactor process in a riser of a fluidized bed reactor and a regeneration process in a regenerator, such that the stability and the catalytic cracking efficiency of the catalytic cracking catalyst may be more excellent. In some embodiments, Expression 1 may be 2<(D90−D10)/D50<4, or Expression 1 may be 2<(D90−D10)/D50<3.

The method for converting waste plastic pyrolysis oil into light olefins with a high yield of the present disclosure will be more specifically described. In the step (1) of putting the waste plastic pyrolysis oil into the reactor, the reactor may be a fluidized bed reactor. In a case where a fixed bed reactor is used, although a yield of olefins is high at the beginning of the reaction in which hydrocarbons are in contact with the catalytic cracking catalyst, deactivation of the catalyst and excessive production of coke occur over time, such that a conversion rate of hydrocarbons and the yield of olefins are reduced as a whole, and a lot of energy is consumed in a regeneration process. Using a fluidized bed reactor may be preferable in terms of a catalyst regeneration process described below as well as solving the above problems. In addition, improved light olefin conversion efficiency may be realized. In this case, the fluidized bed reactor may be a riser.

In an exemplary embodiment, the waste plastic pyrolysis oil may comprise a vacuum gas oil (VGO) component having a boiling point of 340° C. or higher at atmospheric pressure. In the related art, VGO has conversion efficiency that is much lower than those of Kerosene and Naphtha in the light olefin conversion process, making it difficult to use VGO, and therefore, the conversion process has been performed mainly using Kerosene and Naphtha as a feedstock excluding VGO. On the other hand, the light olefin high-yield conversion process of the present disclosure may implement a significantly excellent cracking efficiency and light olefin conversion efficiency even when waste plastic pyrolysis oil comprising a vacuum gas oil (VGO) component is used as a feedstock.

The step (2) of allowing the waste plastic pyrolysis oil to react in the reactor in the presence of the catalytic cracking catalyst comprising the compound of the alkali metal or the compound of the alkaline earth metal and a zeolite may be performed at a temperature of 400 to 600° C. and a reaction pressure of 50 to 200 kPa. Since the reaction efficiency in the step (2) is highly dependent on the temperature and pressure, the energy consumption may be minimized under the above conditions, and the catalytic deactivation may be effectively suppressed. In some embodiments, the temperature may be 400 to 550° C. and the pressure may be 50 to 150 kPa, or the temperature may be 400 to 500° C. and the pressure may be 50 to 100 kPa.

In addition, the reaction efficiency in the step (2) may be dependent on a plateau time, a catalyst/pyrolysis oil ratio, or a pyrolysis oil/steam ratio. The plateau time may be about 0.1 to 600 seconds, the catalyst/pyrolysis oil ratio may be 1 to 50, and the pyrolysis oil/steam ratio may be 0.01 to 10, or the plateau time may be about 0.5 to 120 seconds, the catalyst/pyrolysis oil ratio may be 5 to 30, and the pyrolysis oil/steam ratio may be 0.1 to 2.0, or the plateau time may be about 1 to 20 seconds, the catalyst/pyrolysis oil ratio may be 10 to 20, and the pyrolysis oil/steam ratio may be 0.3 to 1.

After performing the catalytic cracking reaction, light olefins may be recovered by separating a reaction product into the catalytic cracking catalyst and oil through the step (3). In some embodiments, a reaction product obtained in the step (2) may be introduced into a cyclone described below, the catalytic cracking catalyst and oil may be separated within a short time, and light olefins may be recovered from the oil.

In an exemplary embodiment, the method for converting waste plastic pyrolysis oil into light olefins with a high yield may further comprise: (3-1) recovering the catalytic cracking catalyst separated in the step (3), putting the recovered catalytic cracking catalyst into a regenerator, and then oxidizing the compound of the alkali metal or oxidizing the compound of the alkaline earth metal to form an oxidized compound of the alkali metal or an oxidized compound of the alkaline earth metal; and (3-2) circulating the catalytic cracking catalyst comprising the oxidized compound of an alkali metal or the oxidized compound of the alkaline earth metal to the step (2) and re-feeding the catalytic cracking catalyst comprising the oxidized compound of the alkali metal or the oxidized compound of the alkaline earth metal into the reactor. As the separated catalytic cracking catalyst is put into the regenerator, the compound of the alkali metal or the compound of the alkaline earth metal is oxidized in the presence of oxygen gas, the catalytic cracking catalyst comprising the oxidized compound of the alkali metal or the oxidized compound of the alkaline earth metal is circulated to the step (2) again, and the catalytic cracking catalyst comprising the oxidized compound of the alkali metal or the oxidized compound of the alkaline earth metal is re-fed into the reactor, economic feasibility may be improved, and impurities such as chlorine and/or nitrogen contained in the waste plastic pyrolysis oil may be removed. In some embodiments, the reactor may be a riser of a fluidized bed reactor, and the regenerator may be a regenerator of a fluidized bed reactor. The impurities are bound to and decomposed by the catalytic cracking catalyst comprising the compound of the alkali metal or the compound of the alkaline earth metal, and may be continuously discharged in a gaseous state in the process, thereby removing the impurities. In addition, the method for converting waste plastic pyrolysis oil into light olefins with a high yield further comprises the step (3-1) and the step (3-2), such that a heat loss or energy loss generated in a process of removing a waste catalyst and making-up a new catalytic cracking catalyst in the related art may be solved.

In an exemplary embodiment, the step (3-1) may be performed at 600 to 800° C. in the presence of oxygen gas. The oxidation process may be performed by additionally injecting oxygen gas into the regenerator, and the reaction efficiency may be the most excellent in the above temperature range. In some embodiments, the temperature may be 600 to 750° C., or 600 to 700° C.

The process of adsorbing and decomposing the impurities comprised in the waste plastic pyrolysis oil by the compound of the alkali metal or the compound of the alkaline earth metal comprised in the catalytic cracking catalyst in the light olefin conversion process according to an exemplary embodiment of the present disclosure will be specifically described. For example, when the compound of the alkaline earth metal is magnesium oxide, chlorine (Cl) impurities may be adsorbed and decomposed by the magnesium oxide through reactions such as the following Reaction Formulas 1 and 2.

MgO+2HCl→MgCl₂+H₂ [Reaction Formula 1]

2MgCl₂+O₂→2MgO+2Cl₂ [Reaction Formula 2]

Nitrogen impurities may be adsorbed and decomposed by magnesium oxide through the following Reaction Formulas 3 and 4.

MgO+NO₂→MgNO₃ [Reaction Formula 3]

2MgNO₃+7H₂→2MgO+2NH₃+4H₂O [Reaction Formula 4]

Sulfur impurities may be adsorbed and decomposed by magnesium oxide through the following Reaction Formulas 5 to 8.

MgO+SO₂+½O₂→MgSO₄ [Reaction Formula 5]

MgSO₄+4H₂→MgO+H₂S+3H₂O [Reaction Formula 6]

MgSO₄+4H₂→MgS+4H₂O [Reaction Formula 7]

MgS+H₂O→MgO+H₂S [Reaction Formula 8]

In an exemplary embodiment, the method for converting waste plastic pyrolysis oil into light olefins with a high yield may further comprise, before the step (3-1), stripping the catalytic cracking catalyst separated in the step (3). Since the catalytic cracking catalyst separated in the step (3) may comprise unseparated hydrocarbon oils, the method further comprises the stripping step for removing the unseparated hydrocarbon oils, such that the regeneration efficiency may be improved.

As an exemplary embodiment, the catalytic cracking catalyst may be a composite catalyst obtained by mixing the compound of the alkali metal or the compound of the alkaline earth metal, a zeolite, clay, and a binder, and then performing molding. Such a composite catalyst may realize an excellent impurity removal effect by modifying the entire catalytic cracking catalyst with the compound of the alkali metal or the compound of the alkaline earth metal.

As another exemplary embodiment, the catalytic cracking catalyst may be a binary composite catalyst in which a molded catalyst comprising a zeolite, clay, and a binder, a molded catalyst of the compound of the alkali metal or the compound of the alkaline earth metal, and a structure thereof are mixed. As described above, the compound of the alkali metal or the compound of the alkaline earth metal may have a structure. For example, the compound of the alkali metal or the compound of the alkaline earth metal may have a hydrotalcite-like structure, a double-layer hydroxide structure, a spinel structure, or a perovskite structure. Such a binary composite catalyst has excellent regeneration efficiency and may maintain catalytic activity for a long time.

As an exemplary embodiment, a method for preparing a catalytic cracking catalyst may be as follows. The method for preparing a catalytic cracking catalyst may comprise: preparing a mixed solid fine powder by mixing and crushing a zeolite, clay, and the compound of the alkali metal or the compound of the alkaline earth metal; preparing a binder comprising alumina gel; preparing a mixed slurry by uniformly mixing the mixed solid fine powder and the binder; preparing a spherical catalyst by spray-drying the mixed slurry and then performing calcination; recovering a catalyst having an average particle size of 5 to 200 μm through sieving; and ion-exchanging the recovered catalyst with an aqueous solution of rare earth metal (RE) chloride containing cerium and lanthanum and then performing calcination. The spherical molded catalyst prepared by such a method has an excellent effect of adsorbing impurities by the compound of the alkali metal or the compound of the alkaline earth metal and excellent hydrothermal stability, may effectively protect the acid site of the zeolite, and above all, may convert waste plastic pyrolysis oil into light olefins with a relatively high yield during catalytic cracking. In addition, the catalytic cracking catalyst may exhibit high cracking activity and stability even in a high temperature and high humidity atmosphere.

In an exemplary embodiment, a yield of converting the waste plastic pyrolysis oil into light olefins may be 10% or more. Light olefins may be converted efficiently from waste plastic pyrolysis oil using the catalytic cracking catalyst comprising the compound of the alkali metal or the compound of the alkaline earth metal and a zeolite. In some embodiments, a conversion yield into light olefins may be 25% or more, or 40% or more. Without limitation, the conversion yield into light olefins may be 90% or less.

In an exemplary embodiment, in the light olefins converted from the waste plastic pyrolysis oil, a content of chlorine may be 10 ppm or less and/or a content of nitrogen may be 50 ppm or less. The process is performed using the catalytic cracking catalyst comprising a compound of the alkali metal or the compound of the alkaline earth metal and a zeolite, such that light olefins with minimized impurities may be obtained. The compound of the alkali metal or the compound of the alkaline earth metal may effectively remove chlorine because it has the most excellent ability to trap chlorine among the impurities. In addition, the compound of the alkali metal or the compound of the alkaline earth metal may also reduce impurities such as nitrogen and/or sulfur, and specifically, in the converted light olefins, the content of chlorine and/or the content of nitrogen may be 5 ppm or less and 30 ppm or less, respectively, or the content of chlorine and the content of nitrogen may be 3 ppm or less and 10 ppm or less, respectively. Without limitation, the content of chlorine and the content of nitrogen may be 0.01 ppm or more and 0.1 ppm or more, respectively.

In addition, the present disclosure provides a device for converting waste plastic pyrolysis oil into light olefins with a high yield, the device comprising: a fluidized bed reactor into which waste plastic pyrolysis oil is introduced and in which a catalytic cracking reaction is performed in the presence of a catalytic cracking catalyst comprising (a) a compound of an alkali metal or a compound of an alkaline earth metal and (b) a zeolite to form a product; a cyclone into which the product is introduced from the fluidized bed reactor and in which the product is separated into the catalytic cracking catalyst and oil; and a stabilizer into which the oil is introduced from the cyclone and in which the oil is separated into a gas component and a liquid component.

In an exemplary embodiment, the alkali metal may comprise a metal selected from lithium, sodium, potassium, and/or rubidium, and the alkaline earth metal may comprise a metal selected from magnesium, calcium, strontium, and/or barium. Specifically, considering the ability to trap impurities, the alkali metal may be sodium or potassium, and the alkaline earth metal may be magnesium or calcium.

In an exemplary embodiment, the compound of the alkali metal or the compound of the alkaline earth metal may comprise an oxide, a hydroxide, a carbonate, a silicate, an acetate, a nitride, or an alkoxide of the alkali metal or the alkaline earth metal. As another exemplary embodiment, the compound of the alkali metal or the compound of the alkaline earth metal may have a structure. For example, the compound of the alkali metal or the compound of the alkaline earth metal may have a hydrotalcite-like structure, a double-layer hydroxide structure, a spinel structure, or a perovskite structure. This is merely a non-limiting example, and the present disclosure is not limited thereto. The compound of the alkali metal or the compound of the alkaline earth metal described above may prevent deactivation of the catalytic cracking catalyst by trapping impurities contained in the waste plastic pyrolysis oil, such as chlorine, nitrogen, sulfur, or oxygen, and may minimize impurities in the converted light olefins.

Preferably, considering the light olefin conversion efficiency and impurity removal efficiency of the waste plastic pyrolysis oil, the compound of the alkali metal or the compound of the alkaline earth metal may be potassium oxide, calcium oxide, or magnesium oxide, or magnesium oxide. The magnesium oxide has the best ability to trap chlorine among the impurities, such that the magnesium oxide may most effectively remove chlorine.

The waste plastic pyrolysis oil is fed into the fluidized bed reactor, and may be fed after being heated to a temperature of 30 to 600° C. for a smoother reaction. The waste plastic pyrolysis oil may be mixed with the catalytic cracking catalyst comprising the compound of the alkali metal or the compound of the alkaline earth metal and a zeolite provided in a fluidized bed reactor, or may be mixed with the catalytic cracking catalyst to be fed from the regenerator through a recirculation line connected to a fluidized bed reactor. In addition, the mixing process of the feedstock and the catalytic cracking catalyst may be configured in various ways known in the art, and all of these configurations fall into the scope of the present disclosure.

In the fluidized bed reactor, a catalytic cracking reaction may be performed at a temperature of 400 to 600° C. and a pressure of 50 to 200 kPa, and in this case, the fluidized bed reactor may be a riser. Therefore, the product may be introduced into the cyclone and may be separated into oil and the catalytic cracking catalyst. The separated oil is introduced into the stabilizer from the cyclone, and may be separated into a gas component and a liquid component through cooling.

In an exemplary embodiment, the device for converting waste plastic pyrolysis oil into light olefins with a high yield may further comprise a regenerator into which the catalytic cracking catalyst is introduced from the cyclone and in which the compound of the alkali metal or the compound of the alkaline earth metal is oxidized to form an oxidized compound of the alkali metal or an oxidized compound of the alkaline earth metal, and the catalytic cracking catalyst comprising the oxidized compound of the alkali metal or the oxidized compound of the alkaline earth metal may be re-introduced into the fluidized bed reactor from the regenerator through a recirculation line. The separated catalytic cracking catalyst may be accumulated in the cyclone, and a stripping steam may be fed through a separate line connected to the bottom of the cyclone. The stripping steam removes and discharges an unseparated hydrocarbon reaction product comprised in the catalytic cracking catalyst while moving upward along the cyclone. The catalytic cracking catalyst that has passed through the stripping steam in the cyclone is introduced into the regenerator, and in this case, impurities such as chlorine and/or nitrogen comprised in the waste plastic pyrolysis oil are adsorbed to the compound of the alkali metal or the compound of the alkaline earth metal comprised in the catalytic cracking catalyst. Gas such as air containing oxygen is introduced into the regenerator through a separate inlet, the catalytic cracking catalyst and oxygen react with each other at a high temperature of 600° C. or higher, and as a result, the impurities adsorbed to the compound of the alkali metal or the compound of the alkaline earth metal may be removed by being discharged as a gas. The catalytic cracking catalyst treated as described above may be re-introduced into the reactor through the recirculation line.

A method for reducing greenhouse gas emissions by converting waste plastic pyrolysis oil into light olefins is provided, comprising: (1) putting waste plastic pyrolysis oil into a reactor; (2) allowing the waste plastic pyrolysis oil to react in the reactor in the presence of a catalytic cracking catalyst comprising: (a) a compound of an alkali metal or a compound of an alkaline earth metal; and (b) zeolite to form a product; and (3) recovering light olefins by separating the catalytic cracking catalyst and oil from the product obtained in step (2).

Hereinafter, the present disclosure will be described in detail with reference to Examples. However, these Examples are intended to describe the present disclosure in more detail, and the scope of the present disclosure is not limited by the following Examples.

### Example 1

### 1) Feedstock

200 g of waste plastics were fed to a batch pyrolysis reactor, and pyrolysis was performed at 500° C., thereby obtaining pyrolysis oil. The distribution of the mixture of hydrocarbon oils included in the pyrolysis oil is shown in Table 1.

TABLE 1

| | Predictive | Boiling | |
|---|---|---|---|
| Cut Name | carbon range | point (° C.) | wt % |
| Naphtha | C7 to C9 | 80 to 150 | 22.1 |
| KEROSENE | C10 to C17 | 150 to 265 | 28.1 |
| LGO | C18 to C20 | 265 to 340 | 15.9 |
| VGO | C21 or more | >340 | 33.9 |
| Sum | — | — | 100 |

The pyrolysis oil was separated by boiling point through a distiller, and only C21+VGO was selectively recovered, thereby preparing a pyrolysis oil feedstock. In the impurities in the recovered VGO, the contents of chlorine, nitrogen, and sulfur were 16 ppm, 265 ppm, and 23 ppm, respectively.

### 2) Preparation of Catalyst

70 g of Pseudo-boehmite was introduced into 500 g of water, 10 g of formic acid was introduced while stirring the mixture at room temperature, and then the mixture was maintained for 3 hours, thereby preparing alumina gel. Clay(Kaoline), ZSM-5, and MgO were introduced into a mixer so that an Mg/Al molar ratio was 25 and then stirring was performed for 10 minutes. The amount of MgO introduced was 5 wt % of the total weight of clay and ZSM-5. After the mixture of clay, ZSM-5, and MgO was stirred in the mixer to make the mixture into uniform small particles, the alumina gel was introduced, and then stirring was performed again with the mixer. 60 g of Ludox (AS 40) monodispersed ammonium stabilized colloidal silica was introduced during stirring, and then a viscosity of the slurry was measured using a viscometer.

Thereafter, in the process of converting the viscosity of the slurry from sol to gel, the slurry was prepared into a spherical catalyst through spray drying, the spherical catalyst was dried in an oven at 120° C. for 12 hours, and then the dried spherical catalyst was calcined at 550° C. for 3 hours, thereby preparing a fluidized bed catalyst. The recovered catalyst was separated by screening only a catalyst having a particle size of 30 to 200 μm through sieving, the screened catalyst was subjected to ion-exchange with a 5% RE-metal solution at 60° C. for 3 hours, the catalyst was dried, and then the dried catalyst was calcined at 550° C. for 3 hours, thereby preparing a final catalyst.

### 3) Catalytic Cracking Process

The catalytic activity was evaluated by operating a Davison circulating riser (DCR) reaction system for 120 minutes. A cracking reaction and a catalyst regeneration reaction in a regenerator were performed at 530° C. and 730° C., respectively, and the reaction was performed while a stripper where a cracking reaction occurred was maintained at 527° C. A feedstock and steam were introduced into a reaction unit at 850 g/h and 80 g/h, respectively, N₂ was introduced at a total rate of 220 liters per second (lps) by introducing N₂ into a stripper at 180 lps and into a reactor at 40 lps, and the catalyst separated in a cyclone was introduced into the regenerator to be regenerated. Air was introduced at a rate of 900 lps for regeneration of the catalyst in the regenerator. The operation was performed under a condition in which a Cat/Oil ratio, which was a ratio of the catalyst to the feedstock introduced, was set to 11 to 20. A product was separated into a gas and a liquid in a stabilizer, and then the recovered gas was analyzed through GC, and the liquid was analyzed through SIMDIST. Coke, CO₂, and H₂ were analyzed by a CO/CO₂ analyzer.

### Example 2

A process was performed under the same conditions as those of Example 1, except that a catalyst was prepared so that the Mg/Al molar ratio of the catalytic cracking catalyst was 50.

### Example 3

A process was performed under the same conditions as those of Example 1, except that a catalyst was prepared using hydrotalcite (Mg₆Al₂CO₃(OH)₁₆·4H₂O) instead of MgO in the preparation of the catalyst.

### Example 4

A process was performed under the same conditions as those of Example 1, except that a catalyst was prepared using MgSiO₃ instead of MgO.

### Example 5

A process was performed under the same conditions as those of Example 1, except that a catalyst was prepared using CaO instead of MgO.

### Example 6

A process was performed under the same conditions as those of Example 1, except that a catalyst was prepared using K₂O instead of MgO.

### Example 7

A process was performed under the same conditions as those of Example 1, except that a regenerator was not used and a regeneration process of the catalyst was not performed.

### Comparative Example 1

A process was performed under the same conditions as those of Example 1, except that a catalyst was prepared without using MgO.

### Evaluation Examples

A Davison circulating riser (DCR) reaction system was operated for 120 minutes, and analysis was performed on the produced oil. A gas component oil was quantified through on-line gas chromatography (model name HP 6890N), and a liquid component oil was recovered in a storage tank and then quantified through SIMDIST.

The contents (ppm) of chlorine in the finally obtained light olefins were evaluated by measurement through IC and TNS analysis methods.

The analysis results are shown in Table 2.

TABLE 2

| | | | | | | | Example 7 | |
|---|---|---|---|---|---|---|---|---|
| | | | | | | | (Generation | Comparative |
| | Example 1 | Example 2 | Example 3 | Example 4 | Example 5 | Example 6 | process X) | Example 1 |
| Catalyst | MgO, | MgO, | Mg₆Al₂CO₃(OH)₁₆•4H₂O, | MgSiO₃, | CaO, | K₂O, | MgO, | Zeolite, |
| composition | zeolite, | zeolite, | zeolite, | zeolite, | zeolite, | zeolite, | zeolite, | clay, binder |
| | clay, binder | clay, binder | clay, binder | clay, binder | clay, binder | clay, binder | clay, binder | |
| Mg/Al molar | 25 | 50 | 25 | 25 | — | — | 25 | — |
| ratio | | | | | | | | |
| Coke | 6.7 | 11.2 | 5.9 | 14.6 | 7.5 | 14.7 | 15.6 | 16.5 |
| (wt %) | | | | | | | | |
| LCO + Naptha + C | 87 | 76.2 | 86.7 | 72.3 | 81.8 | 72.1 | 65.6 | 60.8 |
| 4- oil | | | | | | | | |
| (wt %) | | | | | | | | |
| Light olefins | 45.7 | 26.1 | 29.7 | 12.2 | 39.6 | 11.2 | 10.1 | 8 |
| (ethylene + | | | | | | | | |
| propylene + | | | | | | | | |
| butylene + | | | | | | | | |
| butadiene) | | | | | | | | |
| (wt %) | | | | | | | | |
| Chlorine in | <1 | 4 | 2 | 6 | 3 | 5 | 9 | 14 |
| light olefins | | | | | | | | |
| (ppm) | | | | | | | | |

As shown in Table 2, in Examples 1 to 7 in which the catalytic cracking catalyst containing a compound of an alkali metal or an alkaline earth metal and a zeolite was used, the cracking efficiency, the light olefin conversion yield, and the chlorine reduction effect were excellent compared to those in Comparative Example 1.

In Example 1, as the catalytic cracking catalyst containing MgO and satisfying an Mg/Al molar ratio of 25 was used, the cracking efficiency, the light olefin conversion yield, and the chlorine reduction effect were most excellent.

In Example 2, as the catalytic cracking catalyst satisfying an Mg/Al molar ratio of 50 was used, the cracking efficiency, the light olefin conversion yield, and the chlorine reduction effect were slightly deteriorated than those in Example 1, but were excellent compared to those in Comparative Example 1.

In Example 3, as the catalytic cracking catalyst containing hydrotalcite (Mg₆Al₂CO₃ (OH)₁₆·4H₂O) rather than MgO was used, the coke reduction effect was excellent compared to that in Example 1.

In Example 4, as the catalytic cracking catalyst containing MgSiO₃ rather than MgO was used, the cracking efficiency, the light olefin conversion yield, and the chlorine reduction effect were slightly deteriorated than those in Example 1, but were excellent compared to those in Comparative Example 1.

In Example 5, as the catalytic cracking catalyst containing CaO rather than MgO was used, the cracking efficiency and the light olefin conversion yield were second only to those in Example 1.

In Example 6, as the catalytic cracking catalyst containing K₂O rather than MgO was used, the cracking efficiency, the light olefin conversion yield, and the chlorine reduction effect were slightly deteriorated than those in Example 1, but were excellent compared to those in Comparative Example 1.

In Example 7, as the regeneration process of the catalytic cracking catalyst was omitted, the cracking efficiency, the light olefin conversion yield, and the chlorine reduction effect were slightly deteriorated than those in Example 1, but were excellent compared to those in Comparative Example 1.

In Comparative Example 1, as the catalytic cracking catalyst that did not contain a compound of an alkali metal or an alkaline earth metal was used, the cracking efficiency, the light olefin conversion yield, and the chlorine reduction effect were the lowest.

As set forth above, light olefins may be obtained from waste plastic pyrolysis oil with a high yield by the method for converting waste plastic pyrolysis oil into light olefins with a high yield according to the present disclosure.

The method for converting waste plastic pyrolysis oil into light olefins with a high yield according to the present disclosure may implement a conversion yield of 10% or more, or 25% or more, or 40% or more.

The method for converting waste plastic pyrolysis oil into light olefins with a high yield according to the present disclosure may stably perform a light olefin conversion process for a long time by preventing deactivation of the catalytic cracking catalyst due to impurities contained in the waste plastic pyrolysis oil using a fluidized bed reactor.

The method for converting waste plastic pyrolysis oil into light olefins with a high yield according to the present disclosure may minimize the content of impurities such as chlorine or nitrogen in the converted light olefins to several ppm.

Although exemplary embodiments of the present disclosure have been described hereinabove, the present disclosure is not limited to the exemplary embodiments, but may be implemented in various different forms, and it will be apparent to those skilled in the art to which the present disclosure pertains that the exemplary embodiments may be implemented in other specific forms without departing from the technical idea or essential feature of the present disclosure. Therefore, it is to be understood that the exemplary embodiments described hereinabove are illustrative rather than restrictive in all aspects.

What is claimed is:

1. A method for converting waste plastic pyrolysis oil into light olefins with a high yield, the method comprising:
(1) putting waste plastic pyrolysis oil into a reactor;
(2) allowing the waste plastic pyrolysis oil to react in the reactor in the presence of a catalytic cracking catalyst comprising: (a) a compound of an alkali metal or a compound of an alkaline earth metal; and (b) zeolite to form a product; and
(3) recovering light olefins by separating the catalytic cracking catalyst and oil from the product obtained in step (2).

2. The method of claim 1, wherein the compound of the alkali metal or the compound of the alkaline earth metal comprises an oxide, a hydroxide, a carbonate, a silicate, a sulfate, an acetate, a nitride, or an alkoxide.

3. The method of claim 1, wherein the compound of the alkaline earth metal comprises magnesium oxide.

4. The method of claim 3, wherein the catalytic cracking catalyst satisfies an Mg/Al molar ratio of 10 to 40.

5. The method of claim 1, wherein the zeolite comprises ZSM-5, ZSM-11, Y-zeolite, ferrierite, mordenite, MCM-22, SUZ-4, and/or L-type zeolite.

6. The method of claim 1, wherein the catalytic cracking catalyst further comprises clay and a binder.

7. The method of claim 6, wherein the binder comprises a silica-based compound and/or an aluminum-based compound.

8. The method of claim 6, wherein the catalytic cracking catalyst comprises 1 to 10 wt % of the compound of the alkali metal or the compound of the alkaline earth metal, 20 to 70 wt % of the zeolite, 10 to 60 wt % of the clay, and 10 to 50 wt % of the binder.

9. The method of claim 1, wherein the catalytic cracking catalyst has an average particle size of 50 to 2,000 μm.

10. The method of claim 1, wherein the waste plastic pyrolysis oil comprises a vacuum gas oil (VGO) component having a boiling point of 340° C. or higher at atmospheric pressure.

11. The method of claim 1, wherein step (2) is performed at a temperature of 400 to 600° C. and a reaction pressure of 50 to 200 kPa.

12. The method of claim 1, further comprising:
(3-1) recovering the catalytic cracking catalyst separated in step (3), putting the recovered catalytic cracking catalyst into a regenerator, and then oxidizing the compound of the alkali metal or the alkaline earth metal to form an oxidized compound of the alkali metal or an oxidized compound of the alkaline earth metal; and
(3-2) circulating the catalytic cracking catalyst comprising the oxidized compound of the alkali metal or the an oxidized compound of the alkaline earth metal to step (2) and re-feeding the catalytic cracking catalyst comprising the oxidized compound of the alkali metal or the oxidized compound of the alkaline earth metal into the reactor.

13. The method of claim 12, wherein step (3-1) is performed at 600 to 800° C. in the presence of oxygen gas.

14. The method of claim 12, further comprising, before step (3-1), stripping the catalytic cracking catalyst separated in step (3).

15. The method of claim 1, wherein in the recovered light olefins, a content of chlorine is 10 ppm or less and a content of nitrogen is 50 ppm or less.

16. A device for converting waste plastic pyrolysis oil into light olefins with a high yield, the device comprising:
a fluidized bed reactor into which waste plastic pyrolysis oil is introduced and in which a catalytic cracking reaction is performed in the presence of a catalytic cracking catalyst comprising (a) a compound of an alkali metal or a compound of an alkaline earth metal and (b) a zeolite to form a product;
a cyclone into which the product is introduced from the fluidized bed reactor and in which the product is separated into the catalytic cracking catalyst and oil; and
a stabilizer into which the oil is introduced from the cyclone and in which the oil is separated into a gas component and a liquid component.

17. The device of claim 16, wherein the compound of an alkali metal or an alkaline earth metal comprises an oxide, a hydroxide, a carbonate, a silicate, a sulfate, an acetate, a nitride, or an alkoxide.

18. The device of claim 16, wherein the compound of the alkaline earth metal comprises magnesium oxide.

19. The device of claim 16, further comprising a regenerator into which the catalytic cracking catalyst is introduced from the cyclone and in which the compound of the alkali metal or the compound of the alkaline earth metal is oxidized to form an oxidized compound of the alkali metal or an oxidized compound of the alkaline earth metal,
wherein the catalytic cracking catalyst comprising the oxidized compound of the alkali metal or the oxidized compound of the alkaline earth metal is re-introduced into the fluidized bed reactor from the regenerator through a recirculation line.

20. A method for reducing greenhouse gas emissions by converting waste plastic pyrolysis oil into light olefins, comprising:
(1) putting waste plastic pyrolysis oil into a reactor;
(2) allowing the waste plastic pyrolysis oil to react in the reactor in the presence of a catalytic cracking catalyst comprising: (a) a compound of an alkali metal or a compound of an alkaline earth metal; and (b) zeolite to form a product; and
(3) recovering light olefins by separating the catalytic cracking catalyst and oil from the product obtained in step (2).
